(19) United States
(12) Patent Application Publication
(10) Pub. No.: US 2023/0040583 A1
(43) Pub. Date: Feb. 9, 2023

(54) Universal Thermal Contact Pad With Twisting Paddles

(71) Applicant: C. R. Bard, Inc., Franklin Lakes, NJ (US)

(72) Inventors: Alexandra A. Falis, Marietta, GA (US); Ronald N. Legaspi, Alpharetta, GA (US); Abigail A. Wilms, Tucker, GA (US); Sudhakar Jagannathan, Alpharetta, GA (US); Jacob A. Bible, Covington, GA (US)

(21) Appl. No.: 17/881,270

(22) Filed: Aug. 4, 2022

Related U.S. Application Data
(60) Provisional application No. 63/230,276, filed on Aug. 6, 2021.

(51) Int. Cl. A61F 7/02; A61F 7/00

(52) U.S. Cl. CPC A61F 7/02; A61F 7/0085; A61F 2007/0024; A61F 2007/0039; A61F 2007/0244; A61F 2007/0269; A61F 2007/0054

(57) ABSTRACT

Disclosed herein is a medical pad for exchanging thermal energy between a targeted temperature management (TTM) fluid and a patient. The pad can include a central pad and a plurality of extension pads coupled with the central pad, where the extension pads extend away from right and left lateral sides of the central pad and where each of the central pad and the plurality of extension pads includes a fluid containing layer configured for circulation of a TTM fluid therein. A method can include removing a portion of the hydrogel liner and placing a remaining portion of the hydrogel liner between the hydrogel layer and the patient. The method can also include rotating and folding one or more of the extension pads to defeat the thermal energy exchange of the rotated and folded extension pads.

### PRIORITY

This application claims the benefit of priority to U.S. Provisional Application No. 63/230,276, filed Aug. 6, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g. water) through one or more thermal contact pads coupled to a patient to affect surface-to-surface thermal energy exchange with the patient. In general, TTM systems include a TTM fluid control module coupled to at least one contact pad via a fluid deliver line. One such thermal contact pad is disclosed in U.S. Pat. No. 6,197,045 titled “Cooling/heating Pad and System” filed Jan. 4, 1999, which is incorporated herein by reference in its entirety.

As these and other medical applications have evolved, the accommodation of different patient sizes has become more important. Disclosed herein are embodiments of devices and methods for the adjusting the patient contact area of the thermal pad to better accommodate patients of different sizes.

### SUMMARY OF THE INVENTION

Briefly summarized, disclosed herein is a medical pad for exchanging thermal energy between a targeted temperature management (TTM) fluid and a patient. The pad includes a central pad defining a top end and a bottom end, where the central pad is configured for placement on a back side of the patient. The pad further includes a plurality of extension pads coupled with the central pad, where the extension pads extend away from right and left lateral sides of the central pad, and each extension pad is coupled with the central pad via a neck extending between the extension pad and the central pad. The central pad may be sized to extend laterally across the back side of the patient and downward along the legs of the patient.

The pad may include a slit, where the slit (i) is disposed centrally between the right and left lateral sides of the central pad, (ii) extends upward away from the bottom end of the central pad, and (iii) extends between a top side and an underside of the central pad.

The pad may include indicia disposed on the top side of the central pad, and the indicia may include an alignment indicium to aide in applying the central pad to the back side of the patient.

The pad further includes a fluid containing layer configured for circulation of a TTM fluid therein, and the fluid containing layer includes at least one delivery flow path and at least one return flow path extending along each neck. The fluid containing layer may include a fluid free zone, where the fluid free zone is disposed centrally between the right and left lateral sides of the central pad, and the fluid free zone extends downward away from the top end of the central pad.

The pad may further include (i) an insulation layer disposed on a top side of the fluid containing layer, (ii) a hydrogel layer disposed on an underside of the fluid containing layer, and (iii) a hydrogel liner coupled with the hydrogel layer on an underside of the hydrogel layer.

In use, the hydrogel layer may be disposed in direct contact with a skin of the patient. The hydrogel liner may include one or more separation lines, where the separation lines (i) extend laterally across the central pad between the right and left lateral sides of the central pad, and (ii) facilitate separation of the hydrogel liner into two or more portions of the hydrogel liner. In use, at least one portion of the hydrogel liner is removed from the pad, and at least one other portion of the hydrogel liner may remain coupled with the pad.

In some embodiments, one or more extension pads are rotatable with respect to the central pad, where the rotation is about an axis of rotation parallel with the neck, and the rotation defines a twist in the neck. One or more extension pads may also be foldable with respect to the central pad, where folding the extension pad defines a crease extending across the neck, and when folded, the extension pad overlaps the central pad. The central pad and/or the one or more extension pads may include an adhesive configured to attach the extension pad to the central pad when the extension pad is folded. In some embodiments, rotating and/or folding the extension pad occludes at least one of the at least one delivery flow path or the at least one return flow path extending along the neck.

Also disclosed herein is a method of providing a targeted temperature management (TTM) therapy to a patient. The method includes providing a thermal contact pad where the thermal contact pad includes a central pad coupled with a plurality of extension pads extending away from right and left lateral sides of the central pad. The method further includes (i) applying the central pad to a back side of the patient, (ii) applying multiple extension pads to a front side of the patient, and (iii) circulating a TTM fluid through the thermal contact pad to facilitate thermal energy exchange with the patient.

The central pad may include a fluid free zone disposed centrally along the central pad, and the method may further include preventing thermal energy exchange with the patient along a spine of the patient.

The method may further include disposing a hydrogel layer of the thermal contact pad in direct contact with a skin of the patient. The thermal contact pad may also include a hydrogel liner coupled with an underside of the hydrogel layer, and the method may further include (i) separating one portion of the hydrogel liner from another portion of the hydrogel liner, (ii) removing the one portion of the hydrogel liner from the hydrogel layer, and (iii) placing the other portion of the hydrogel liner between the hydrogel layer and the patient.

Each of the plurality of extension pads may be coupled with the central pad via a neck, and the method may further include rotating at least one extension pad to define a twist in the neck. The method may further include folding the at least one extension pad to define a crease in the neck. Each neck includes at least one delivery flow path and at least one return flow path extending along the neck, and rotating and/or folding the extension pad may occlude at least one of the at least one delivery flow path or the at least one return flow path.

The method may further include rotating and folding at least two extension pads and the at least two extension pads may include one extension pad extending laterally away from a first side of the central pad, and another extension pad extending laterally away from a second side of the central pad, where the second side is disposed opposite the first side.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a thermal contact pad for employment with a targeted temperature management (TTM) system, in accordance with some embodiments;

FIG. 1B is a cross-sectional side view of a portion the thermal contact pad of FIG. 1A, in accordance with some embodiments;

FIG. 1C is a detailed underside view of a bottom portion the thermal contact pad of FIG. 1A, in accordance with some embodiments;

FIG. 1D is a detailed view of a portion the thermal contact pad of FIG. 1A showing details of extension pads of the thermal contact pad, in accordance with some embodiments;

FIG. 1E illustrates a folded position of the twisted extension pad of FIG. 1D, in accordance with some embodiments;

FIG. 2A illustrates the thermal contact pad of FIG. 1A applied to a back side of a patient according to a first instance of use, in accordance with some embodiments;

FIG. 2B illustrates the thermal contact pad of FIG. 1A further applied to a front side of the patient according to the first instance of use of FIG. 2A, in accordance with some embodiments;

FIG. 3A illustrates the thermal contact pad of FIG. 1A applied to a back side of a patient according to a second instance of use, in accordance with some embodiments; and

FIG. 3B illustrates the thermal contact pad of FIG. 1A further applied to a front side of the patient according to the second instance of use of FIG. 3A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

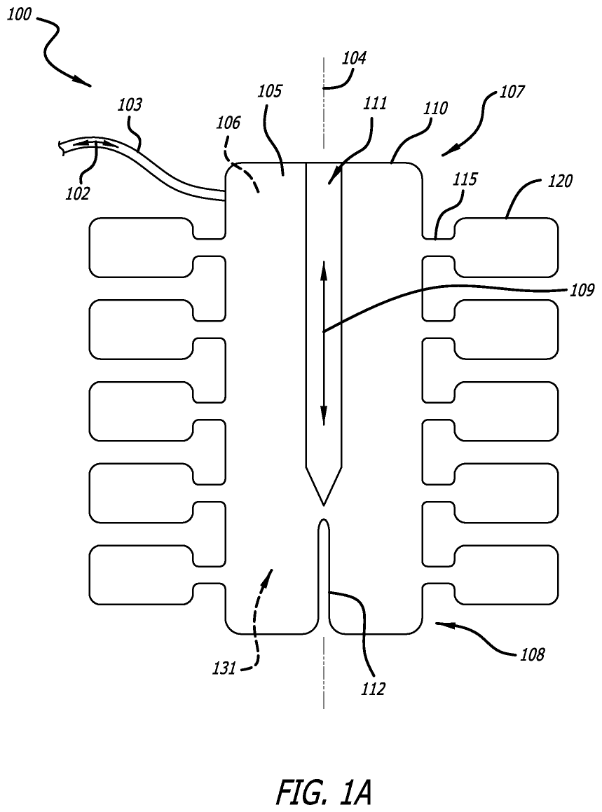

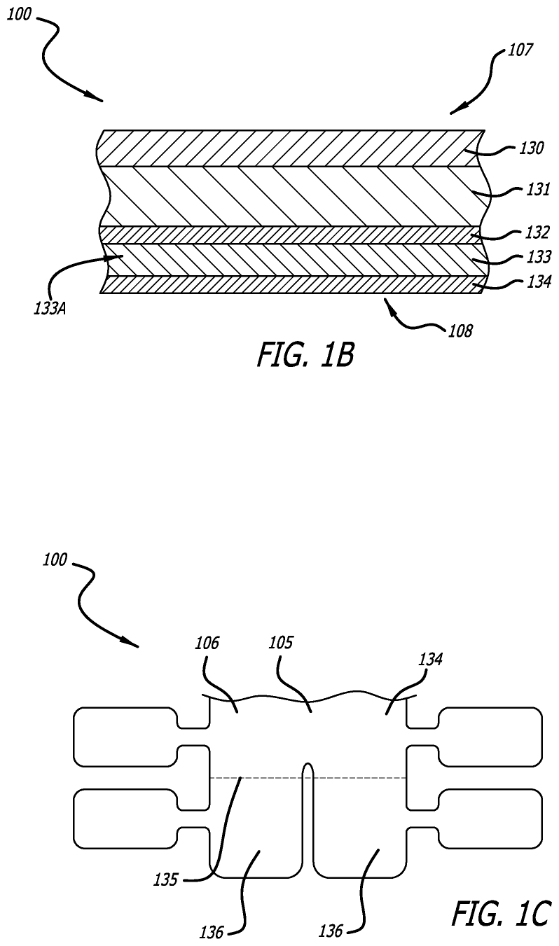

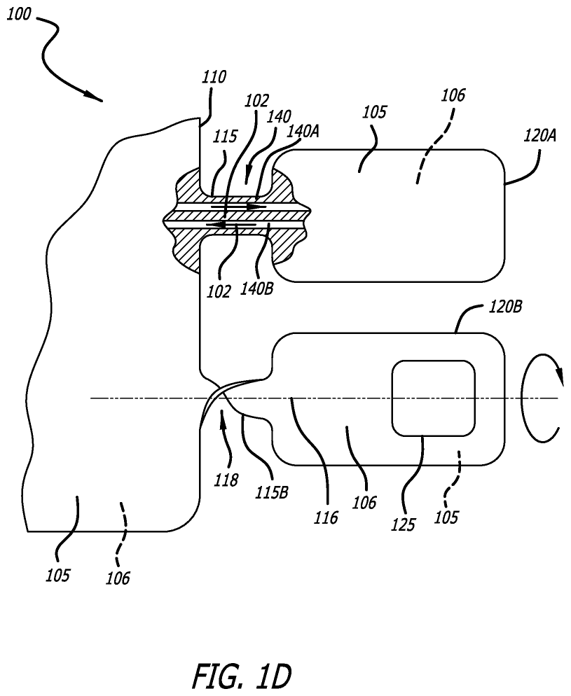

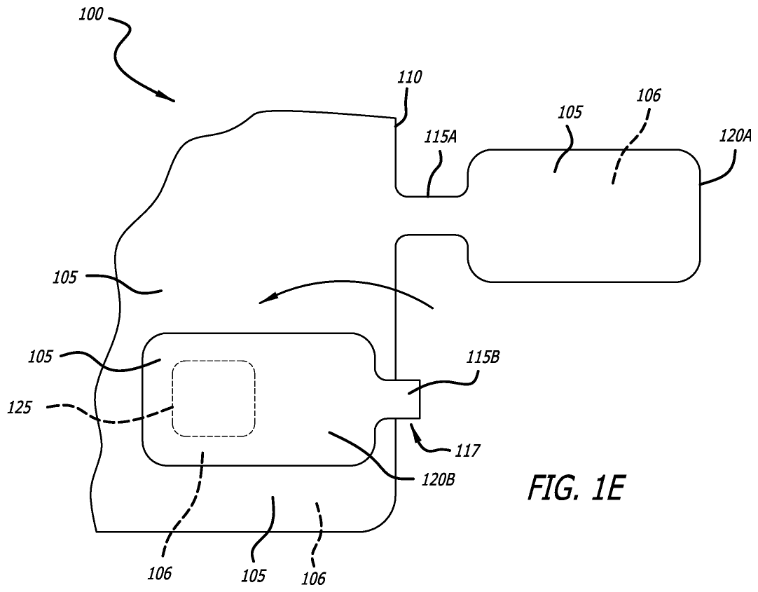

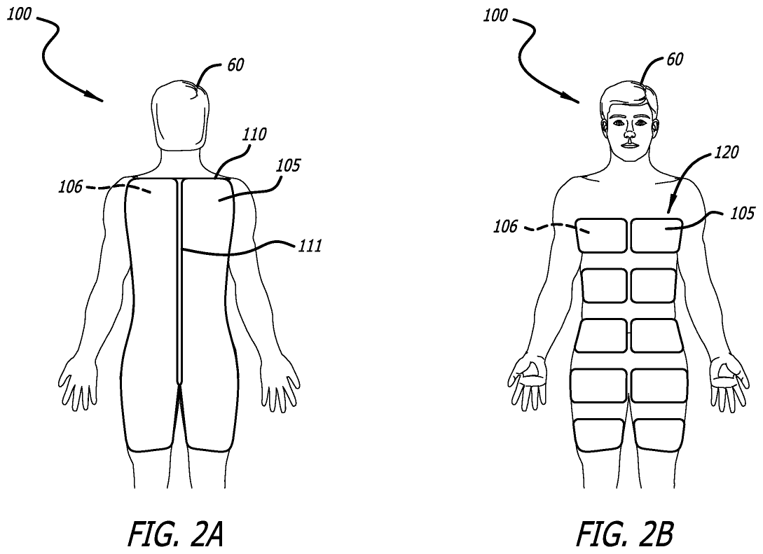

FIG. 1A illustrates a top view of a thermal contact pad for employment with a targeted temperature management (TTM) system, in accordance with some embodiments. The thermal contact pad (pad) 100 is configured to receive a TTM fluid 102 from a TTM system (not shown) via a fluid delivery line 103 and circulate the TTM fluid 102 through a fluid containing layer 131 to facilitate thermal energy exchange between the TTM fluid 102 and a patient (see FIGS. 2A and 2B). The pad 100 generally defines a top side 105 configured to be disposed away from the patient and an underside 106 configured to be disposed in contact with the patient. As described below the pad 100 is configurable to accommodate different patient sizes.

The pad 100 includes a central pad 110. The central pad 110 may generally define a rectangular shape having beveled corners. The central pad 110 extends longitudinally from top end 107 to a bottom end 108. A plurality of extension pads 120 are coupled with and extend away from right and left lateral sides of the central pad 110. In the illustrated embodiment, the pad 100 includes ten pad extensions 120. In other embodiments, the pad 100 may include more or less than ten pad extensions 120. The extension pads 120 may also be rectangular in shape having beveled corners (e.g., having the appearance of a paddle). In other embodiments, the central pad 110 and the extension pads 120 may define shapes other than rectangular such as circular, oval, or a shape that matches or aligns with a shape of the human body. The central pad 110 may define a width generally sized to accommodate an average adult patient. The central pad 110 may further define a length to accommodate a larger/taller than average adult patient. A slit 112 extending along a centerline 104 upward away from the bottom end 108 of the central pad 110 facilities application of the central pad 110 separately to each leg of the patient. The central pad 110 may include indicia 109 to aide in positioning the central pad 110 on the back side of the patient. The indicia 109 may include an alignment indicium to aide in applying the central pad to the back side of the patient.

In use, a clinician may position the pad 100 on the patient so that the central pad 110 is in contact with the back side of the patient's torso from the shoulders to an upper portion of the patient's legs. The clinician may utilize the indicia 109 to align the central pad 100 with the patient's spine. The clinician may adjust the central pad longitudinally so that the slit 112 is adjacent the patient's legs. Once the central pad 110 is positioned, the clinician may wrap the extension pads 120 around the right and left sides of the patient and position the extension pads 120 on the front side of the patient's torso and legs.

A neck 115 couples each extension pad 120 with the central pad 110 as further described below. The position and dimensions of the neck 115 may be configured to accommodate application of the pad 100 to the patient, i.e., provide for an optimal fit of the pad 100 with the patient. For example, the necks 115 of extension pads 120 that are located toward the top end 107 of the central pad 110, may be longer than the necks 115 of extension pads 120 that are located toward the bottom end 108 of the central pad 110, due to the general shape and contours of the human body.

In the illustrated embodiment, each neck 115 is shown centrally located on its respective extension pad 120. In some embodiments, the neck 115 may be positioned toward a top or a bottom of its respective extension pad 120. For example, the necks 115 for extension pads 120 located adjacent the top end 107 of the central pad 110 may be positioned toward the bottom of the extension pads 120 to more comfortably extend under the arms of the patient (see FIG. 2B). Similarly, the necks 115 for extension pads 120 located adjacent the bottom end 108 of the central pad 110 may be positioned toward the bottom of the extension pads 120 to more comfortably extend between the legs of the patient.

The central pad 110 includes a fluid free zone 111 extending longitudinally downward away from a top end 107 of the central pad 110 along the center line 104 of the central pad 110. The fluid containing layer 131 (see FIG. 1B) is discontinued along the fluid free zone 111, thereby defeating thermal energy exchange along the fluid free zone 111. In some instances, it may be beneficial for the patient to prevent thermal energy exchange with a spinal area of the patient. In some embodiments, the indicia 109 or other visible aspects of the central pad 110 may visibly indicate the fluid free zone 111. In use, the clinician may position the central pad 110 on the back side of the patient so that the fluid free zone 111 covers the spine of the patient.

FIG. 1B is a cross-sectional side view of a portion the pad 100, in accordance with some embodiments. The pad 100 includes multiple layers disposed between the top side 105 and the underside 106. Arranged from the top side 105 to the underside 106, the pad includes an insulation layer 130, the fluid containing layer 131, a thermal conduction layer 132, a hydrogel layer 133, and a hydrogel liner 134. The fluid containing layer 131, having TTM fluid 102 circulating therein, defines a heat sink or a heat source for the patient in accordance with a temperature of the TTM fluid 102. The insulation layer 130 thermally isolates the fluid containing layer 131 from the surrounding environment. The thermal conduction layer 132 separates the TTM fluid 102 within the fluid containing layer 131 from a hydrogel 133A within the hydrogel layer 133 and facilitates thermal conduction between the TTM fluid 102 and the hydrogel 133A. The hydrogel layer 133 facilitates thermally intimate contact of the fluid containing layer 131 and the patient. The hydrogel liner 134 is applied to the underside of the hydrogel layer 133 during manufacturing to cover the hydrogel layer 133 and encapsulate the hydrogel 133A. In use, the clinician may remove hydrogel liner 134 from the hydrogel layer 133 to expose the hydrogel 133A, thereby allowing the hydrogel 133A to contact the skin of the patient directly.

FIG. 1C is an underside view of a portion the pad 100, in accordance with some embodiments. There are advantages for matching a size of a patient contact area of the pad 100 with the size of the patient. As such, the pad 100 is configurable to accommodate patients of different sizes. The hydrogel liner 134 may include a separation line such as the perforation line 135 so that segments 136 of the hydrogel liner 134 may be separated from a main portion of the hydrogel liner 134. For example, upon removal of the hydrogel liner 134 from the hydrogel layer 133, the hydrogel liner 134 may be separated from the segments 136 by tearing the hydrogel liner 134 along the perforation line 135 so that the segments 136 may remain in place covering and preventing exposure of the hydrogel 133A (FIG. 1C). As such, the thermal energy exchange of the pad 100 across areas defined by the segments 136 may be effectively defeated. In some embodiments, the hydrogel liner 134 may include multiple perforation lines 135.

In some instances of use, the clinician may determine that the patient is large enough to utilize the entire contract area of the pad 100. In such an instance, the clinician may remove the entire hydrogel liner 134 from the hydrogel layer 133. In other instances, the clinician may determine that the patient is too small to utilize the entire contract area of the pad 100. In such other instances, the clinician may separate one or more portions of the hydrogel liner 134 (e.g., segments 136) from a main portion of the hydrogel liner 134. The clinician may then remove the main portion of the hydrogel liner 134 from the hydrogel layer 133 leaving the separated portions in place covering the hydrogel layer 133. The clinician may then apply to the pad 100 to the patient so that the separated portions (e.g., segments 136) are disposed between the hydrogel layer 133 and the patient.

FIG. 1D is a detailed view of a portion the thermal contact pad 100, in accordance with some embodiments. Shown are exemplary extension pads 120A, 120B coupled with the central pad 110 via necks 115A, 115B, respectively that may represent any or all of the extension pads 120 and the necks 115. The neck 115A is shown cut away to reveal internal fluid pathways 140 extending through the neck 115A. Each neck 115 includes the pathways 140 that provide for transport (i.e., delivery and return) of TTM fluid 102 between the central pad 110 and extension pad 120. In some embodiments, each neck may include more than one delivery flow path 140A and/or more than one return flow path 140B.

One or more of the extension pads 120 may be rotated with respect to the central pad 110 about an axis of rotation 116 defined by the neck 115, thereby causing a twist 118 in the neck 115. In FIG. 1D, the extension pad 120B has been rotated 180 degrees with respect to the central pad 110 while the extension pad 120A remains unrotated. As such, the top side 105 of extension pad 120B is shown facing into the page and the underside 106 of the extension pad 120B is shown facing out of the page. The neck 115B is twisted in accordance with the rotation of the extension pad 120B. In some embodiments, the extension pad 120B may be rotated more or less than 180 degrees.

The extension pad 120 may include an adhesive 125 disposed on the underside 106. In some embodiments, the adhesive 125 may be alternately or additional disposed on the top side 105 of the central pad 110. In some embodiments, the hydrogel liner 134 may be left in place on the underside 106 of the extension pad 120B and the adhesive 125 may be disposed on the hydrogel liner 134.

FIG. 1E illustrates a folded position of the rotated extension pad 120B of FIG. 1D, in accordance with some embodiments. Each extension pad 120 may be folded at the neck 115 forming a crease 117 across the neck 115 so that the pad 120 may be positioned adjacent the top side 105 of the central pad 110. In the illustrated embodiment, the extension pad 120B (which has been rotated 180 degrees) is folded back over the top side 105 of the central pad 110 forming the crease 117. As such, the top sides 105 of both the extension pad 120B and central pad 110 are shown facing out of the page, and the undersides 106 of both the extension pad 120B and central pad 110 are shown facing into the page. The extension pad 120B may be attached to the central pad 110 via the adhesive 125.

The neck 115 may be configured so that when twisted, folded, or both twisted and folded, as shown in FIGS. 1D and 1E, one or both of the internal fluid pathways 140 may become occluded thus preventing the TTM fluid 102 from circulating through the extension pad 120B. In some embodiments, the neck 115B may be configured so that, when subsequently un-folded and/or un-twisted, the internal fluid pathways 140 may become un-occluded so that circulation of the TTM 102 through the extension pad 120B may be restored.

FIGS. 2A and 2B illustrate a first use case of the pad 100, in accordance with some embodiments. FIG. 2A illustrates the thermal contact pad 100 applied to a back side of a patient according to the first use case. In some instances, the patient 60 may be sized to accommodate the entire patient contact area of the pad 100. In the illustrated use case, the entire hydrogel liner 134 (as shown in FIGS. 1B and 1C) has been removed so that the entire area of the underside 106 including the entire hydrogel layer 133 may intimately contact the skin of the patient 60 while the top side 105 faces away from the back side of the patient 60. As shown, the fluid free zone 111 is disposed in alignment with the spine of the patient 60 thus inhibiting thermal energy exchange with the patient 60 along the spine area of the patient 60.

FIG. 2B illustrates the thermal contact pad 100 applied to a front side of a patient 60 according to the first use case. As shown, each of the extension pads 120 are wrapped around the patient and applied to the front side of the patient 60. The underside 106 of each extension pads 120 is in contact with the skin of the patient 60 and the top side 105 of each extension pad 120 faces away from the patient 60.

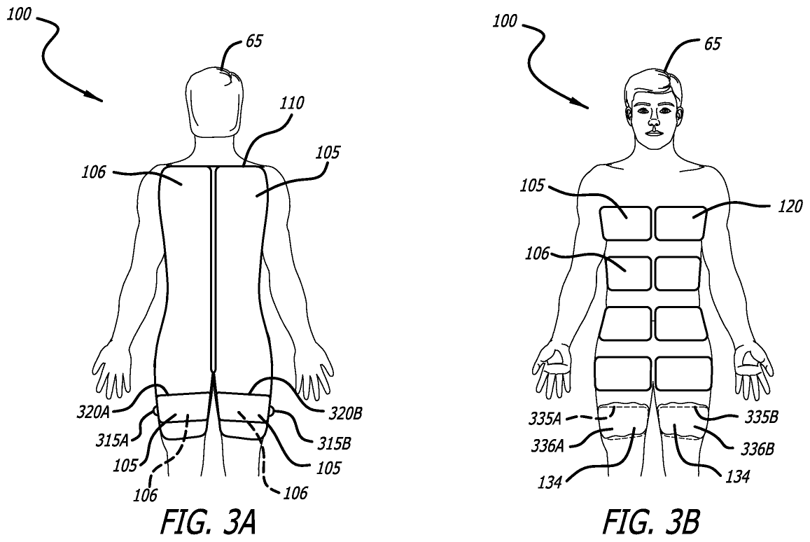

FIG. 3A illustrates the thermal contact pad 100 applied to a back side of a patient 65 according to a second use case, in accordance with some embodiments. In this second use case, the patient 65 is smaller (shorter) that the patient 60 of FIGS. 2A and 2B. In some instances, the patient 65 may be sized too small to accommodate the entire patient contact area of the pad 100. In this second use case, the extension pads 320A, 320B are twisted and folded as shown and described in relation to FIGS. 1D and 1E above. As such, the extension pads 320A, 320B are disposed over the top side 105 of the central pad 110. Due to the twisting and or folding of the necks 315A, 315B, the TTM fluid 102 (see FIG. 1A) may be prevented from circulating through the pad extensions 320A, 320B.

FIG. 3B illustrates the thermal contact pad 100 further applied to a front side of the patient according to the second instance use case. As shown, a subset of the extension pads 120 (i.e., the upper eight extension pads 120 in the illustrated example) are wrapped around the patient 65 and applied to the front side of the patient 65. The underside 106 of each of the subset of extension pads 120 is in contact with the skin of the patient 65 and the top side 105 of each extension pad 120 faces away from the patient 65. As such, thermal energy exchange with the patient 65 of a bottom portion of the pad 100 with a front side of the patient's legs is defeated.

In the second use case, the hydrogel liner segments 336A, 336B have been separated from the main portion of the hydrogel liner 134 at the perforation lines 335A, 335B, respectively. The main portion of the hydrogel liner 134 has been removed from the pad 100 leaving the hydrogel liner segments 336A, 336B attached to the pad 100. In FIG. 3B, a portion of each leg of the patient 65 is shown cut away to reveal the hydrogel liner segments 336A, 336B on the far back side of each leg. The hydrogel liner segments 336A, 336B are disposed between the hydrogel 133A (not shown but see FIG. 1B) and the back side of each leg. As such, thermal energy exchange with the patient 65 of a bottom portion of the pad 100 with a back side of the patient's legs is defeated.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art that, with the aid of the present disclosure, changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A medical pad for exchanging thermal energy with a patient, the pad comprising:
a central pad defining a top end and a bottom end, the central pad configured for placement on a back side of the patient; and
a plurality of extension pads coupled with the central pad, wherein each extension pad extends away from right and left lateral sides of the central pad, and is coupled with the central pad via a neck extending between the extension pad and the central pad, and
wherein each of the central pad and the plurality of extension pads includes a fluid containing layer configured for circulation of a TTM fluid therein.

2. The medical pad according to claim 1, wherein the central pad is sized to extend laterally across the back side of the patient.

3. The medical pad according to claim 1, wherein the central pad is sized to extend downward along the legs of the patient.

4. The medical pad according to claim 1, wherein the central pad comprises a slit, and wherein the slit:
is disposed centrally between the right and left lateral sides of the central pad,
extends upward away from the bottom end of the central pad, and
extends between a top side and an underside of the central pad.

5. The medical pad according to claim 1, wherein:
the central pad comprises indicia disposed on the top side of the central pad, and
the indicia includes an alignment indicium to aide in applying the central pad to the back side of the patient.

6. The medical pad according to claim 5, wherein the fluid containing layer comprises at least one delivery flow path and at least one return flow path extending along each neck.

7. The medical pad according to claim 5, wherein the fluid containing layer comprises a fluid free zone, and wherein the fluid free zone is disposed centrally between the right and left lateral sides of the central pad, and extends downward away from the top end of the central pad.

8. The medical pad according to claim 5, further comprising:
an insulation layer disposed on a top side of the fluid containing layer;
a hydrogel layer disposed on an underside of the fluid containing layer; and
a hydrogel liner coupled with the hydrogel layer on an underside of the hydrogel layer.

9. The medical pad according to claim 8, wherein in use, the hydrogel layer is disposed in direct contact with a skin of the patient.

10. The medical pad according to claim 8, wherein the hydrogel liner comprises one or more separation lines, and wherein the separation lines:
extend laterally across the central pad between the right and left lateral sides of the central pad, and
facilitate separation of the hydrogel liner into two or more portions of the hydrogel liner.

11. The medical pad according to claim 10, wherein in use at least a first portion of the hydrogel liner is removed from the pad, and a second portion of the hydrogel liner remains coupled with the pad.

12. The medical pad according to claim 1, wherein:
one or more extension pads are rotatable with respect to the central pad,
the rotation is about an axis of rotation parallel with the neck, and
the rotation defines a twist in the neck.

13. The medical pad according to claim 1, wherein:
one or more extension pads are foldable with respect to the central pad,
folding the extension pad defines a crease extending across the neck, and
when folded, the extension pad overlaps the central pad.

14. The medical pad according to claim 13, wherein at least one of the central pad or the one or more extension pads comprises an adhesive configured to attach the extension pad to the central pad when the extension pad is folded.

15. The medical pad according to claim 13, wherein at least one of rotating or folding the extension pad occludes at least one of the at least one delivery flow path or the at least one return flow path extending along the neck.

16. A method of providing a targeted temperature management (TTM) therapy to a patient, comprising:
providing a thermal contact pad comprising a central pad coupled with a plurality of extension pads extending away from right and left lateral sides of the central pad, wherein each of the central pad and the plurality of extension pads includes a fluid containing layer configured for circulation of a TTM fluid therein;
applying the central pad to a back side of the patient;
applying multiple extension pads to a front side of the patient; and
circulating the TTM fluid through the fluid containing layer of the thermal contact pad to facilitate thermal energy exchange with the patient.

17. The method according to claim 16, wherein the central pad comprises a fluid free zone disposed centrally along the central pad, the method further comprising:
preventing thermal energy exchange with the patient along a spine of the patient.

18. The method according to claim 16, further comprising disposing a hydrogel layer of the thermal contact pad in direct contact with a skin of the patient.

19. The method according to claim 16, wherein the thermal contact pad comprises a hydrogel liner coupled with an underside of the hydrogel layer, the method further comprising:
separating a first portion of the hydrogel liner from a second portion of the hydrogel liner;
removing the first portion of the hydrogel liner from the hydrogel layer; and
placing the second portion of the hydrogel liner between the hydrogel layer and the patient.

20. The method according to claim 16, wherein each of the plurality of extension pads is coupled with the central pad via a neck extending between the extension pad the central pad, the method further comprising rotating at least one extension pad to define a twist in the neck.

21. The method according to claim 20, further comprising folding the at least one extension pad to define a crease across the neck.

22. The method according to claim 21, wherein:
each neck comprises at least one delivery flow path and at least one return flow path extending along the neck, and
at least one of rotating or folding the extension pad occludes at least one of the at least one delivery flow path or the at least one return flow path.

23. The method according to claim 21, further comprising rotating and folding at least two extension pads.

24. The method according to claim 23, wherein the at least two extension pads comprise a first extension pad extending laterally away from a first side of the central pad, and a second extension pad extending laterally away from a second side of the central pad, the second side disposed opposite the first side.
